(19) United States
(12) Patent Application Publication
(10) Pub. No.: US 2023/0071343 A1
(43) Pub. Date: Mar. 9, 2023

(54) ENERGY-BASED SURGICAL SYSTEMS AND METHODS BASED ON AN ARTIFICIAL-INTELLIGENCE LEARNING SYSTEM

(71) Applicant: Covidien LP, Mansfield, MA (US)

(72) Inventors: Jing Zhao, Superior, CO (US); Christopher T. Brown, Boulder, CO (US); Anjali Dhiman, Commerce City, CO (US)

(21) Appl. No.: 17/794,274

(22) PCT Filed: Jan. 27, 2021
(86) PCT No.: PCT/US2021/015167

§ 371 Date: Jul. 21, 2022

Related U.S. Application Data
(60) Provisional application No. 62/981,609, filed on Feb. 26, 2020.

(51) Int. Cl. G06T 7/00; A61B 18/14

(52) U.S. Cl. CPC G06T 7/0012; A61B 18/1445; A61B 2018/0063

(57) ABSTRACT

A computer-implemented method includes accessing an activation state of an energy-based surgical instrument, accessing at least one image of tissue, accessing control parameter values of a generator configured to provide energy to the energy-based surgical instrument, storing the control parameter values, receiving input information, annotating the stored control parameter values and the at least one image based on the received information, and tagging the annotated control parameter values and the annotated at least one image.

### FIELD

The disclosure relates to energy-based surgical systems and, more particularly, to energy-based surgical systems and methods based on an artificial intelligence learning system.

### BACKGROUND

Surgical instruments are utilized to perform various treatments on tissue structures. A surgical forceps, for example, is a plier-like device which relies on mechanical action between its jaws to grasp, clamp, and constrict tissue. Energy-based surgical forceps utilize both mechanical clamping action and energy to treat tissue, such as coagulate, cauterize, and/or seal tissue.

While surgical instruments such as energy-based surgical forceps are effective at treating tissue, there is continuing interest in improving energy-based surgical systems and methods.

### SUMMARY

The disclosure relates to energy-based surgical systems and methods that use an artificial intelligence learning system. Although portions of the disclosure discuss particular types of energy-based surgical systems, aspects of the disclosure are applicable to other types of energy-based surgical systems not expressly described herein. As used herein, the term “distal” refers to the portion that is being described which is further from a user, while the term “proximal” refers to the portion that is being described which is closer to a user. Further, to the extent consistent, any of the aspects or embodiments described herein may be used in conjunction with any or all of the other aspects or embodiments described herein.

In accordance with aspects of the disclosure, a computer-implemented method for an energy-based surgical system includes accessing an activation state of an energy-based surgical instrument, accessing image(s) of tissue, accessing control parameter values of a generator configured to provide energy to the energy-based surgical instrument, storing the control parameter values, receiving input information, annotating the stored control parameter values and the image(s) based on the received information, and tagging the annotated control parameter values and the annotated image(s).

In an aspect of the disclosure, the instrument activation state may be prior to energy delivery, during energy delivery, and/or after energy delivery.

In yet another aspect of the disclosure, image(s) may be captured prior to energy delivery, during energy delivery, and/or after energy delivery.

In another aspect of the disclosure, in a case where the at least one image is captured prior to energy delivery, the annotation may include a tissue type, a tissue size, and/or field condition.

In a further aspect of the disclosure, in a case where the at least one image is captured during energy delivery, the annotation may include a presence of steam, a presence of smoke, a presence of vapor, and/or jaw closure.

In yet a further aspect of the disclosure, in a case where the at least one image is captured after energy delivery, the annotation may include tissue sticking, bleeding, and/or lateral thermal spread.

In an aspect of the disclosure, the tagging includes providing a tag for training an artificial intelligence learning system and the method further includes training the artificial intelligence learning system based on the stored control parameter values of the generator, the at least one image, and the tag.

In another aspect of the disclosure, the output of the artificial-intelligence learning system may relate to a predicted outcome of applying the energy to tissue based on the stored control parameter values.

In yet another aspect of the disclosure, the artificial intelligence learning system may include a convolutional neural network that processes the at least one image.

In another aspect of the disclosure, the method may further include processing the at least one image, the stored control parameter values, and the annotations by an artificial intelligence learning system to provide an output relating to a configuration of the control parameter values. In such aspects, the method may further include providing an indication to a clinician based on the output, the indication indicating whether to maintain the control parameter values. In a case where the indication indicates not to maintain the control parameter values, adjusted control parameter values for the generator are provided based on the output of the artificial-intelligence learning system.

In an aspect of the disclosure, the method may further include automatically adjusting the control parameters based on the output of the artificial-intelligence learning system and providing an indication to a clinician that the control parameters have been automatically adjusted.

In another aspect of the disclosure, the method may further include outputting the energy to an energy-based surgical instrument, applying the energy to tissue using the energy-based surgical instrument, and receiving information from the clinician relating to an outcome of applying the energy to tissue.

In yet another aspect of the disclosure, the at least one image may include a video image and/or a still image.

In accordance with aspects of the disclosure, an energy-based surgical system includes: an image capturing device configured to capture at least one image of tissue and a generator configured to provide energy to an energy-based surgical instrument. The generator is configured to execute instructions to perform a method including: accessing an activation state of the energy-based surgical instrument, accessing an image of tissue, accessing control parameter values of the control parameters, storing the control parameter values, receiving input information, annotating the stored control parameter values and based on the received information, and tagging the annotated control parameter values and the at least one image.

In an aspect of the disclosure, the instrument activation state may be prior to energy delivery, during energy delivery and/or after energy delivery

In another aspect of the disclosure, the at least one image is captured prior to energy delivery, during energy delivery, and/or after energy delivery.

In yet another aspect of the disclosure, in a case where the image(s) are captured prior to energy delivery, the annotation may include tissue type, tissue size, and/or field condition.

In another aspect of the disclosure, in a case where the at least one image is captured during energy delivery, the annotation may include the presence of steam, presence of vapor, and/or jaw closure.

In a further aspect of the disclosure, in a case where the image(s) may be captured after energy delivery, the annotation may include tissue sticking, bleeding, and/or lateral thermal spread.

In accordance with aspects of the disclosure, a non-transitory storage medium that stores a program causing a computer to execute a method for an energy-based surgical system is presented. The method includes accessing an activation state of an energy-based surgical instrument, accessing image(s) of tissue, accessing control parameter values of a generator configured to provide energy to the energy-based surgical instrument, storing the control parameter values, receiving input information, annotating the stored control parameter values and the image(s) based on the received information, and tagging the annotated control parameter values and the annotated image(s).

### BRIEF DESCRIPTION OF THE DRAWINGS

Various aspects and features of the disclosure are described herein with reference to the drawings wherein:

FIG. 1 is a simplified illustration of a surgical system including an energy-based surgical instrument, a generator, and an endoscope in use during a surgical procedure, in accordance with aspects of the disclosure;

FIG. 2 is a block diagram of an artificial intelligence learning system in accordance with aspects of the disclosure;

FIG. 3 is a block diagram of another artificial intelligence learning system in accordance with aspects of the disclosure;

FIG. 4 is a block diagram of a data record in accordance with aspects of the disclosure;

FIG. 5 is a perspective view of the energy-based surgical instrument and generator of FIG. 1;

FIG. 6 is a block diagram of the generator of FIG. 1;

FIG. 7 is a block diagram of a controller of the generator of FIG. 1;

FIG. 8 is a flowchart of an operation for adjusting generator parameters in accordance with aspects of the disclosure;

FIG. 9 is a diagram illustrating the relationships between a video stream, instrument activation, and instrument sensor data used to train a surgical system;

FIG. 10 is a perspective view of another surgical system including an energy-based surgical instrument and the generator of FIG. 1 in accordance with aspects of the disclosure; and

FIG. 11 is a perspective view of another surgical system including an energy-based (microwave ablation) instrument and a generator having a user interface for displaying and controlling ablation patterns in accordance with aspects of the disclosure.

### DETAILED DESCRIPTION

The disclosure relates to energy-based surgical systems and methods that use an artificial intelligence learning system. Although portions of the disclosure discuss particular types of energy-based surgical systems, aspects of the disclosure are applicable to other types of energy-based surgical systems not expressly described herein.

Energy-based surgical systems may be used, for example, to seal tissue (e.g., vessels). Tissue sealing involves heating tissue to liquefy the collagen and elastin in the tissue so that it reforms into a fused mass with significantly-reduced demarcation between the opposing tissue structures. To achieve a tissue seal without causing unwanted damage to tissue at the surgical site or collateral damage to adjacent tissue, the application of energy to tissue is controlled to control the temperature of tissue during the sealing process. To properly seal tissue, a balance should be sustained during the sealing process between sufficient heating to denature proteins and vaporize fluids and minimize unwanted damage to tissue. In various situations, implementing such a balance to achieve a proper tissue seal may be based on correlations between the energy-based equipment setting values and/or different conditions of the tissue.

As detailed below, and in accordance with aspects of the disclosure, the disclosure involves processing energy-based surgical system information and tissue condition information using an artificial intelligence learning system. Aspects of the disclosure may also use annotations on the energy-based surgical system information for a particular tissue in order to train an artificial intelligence learning system.

As detailed below, and in accordance with aspects of the disclosure, the disclosure involves systems and methods to simultaneously capture endoscopic video/image data along with electrosurgical generator data to provide “context” for each energy-tissue interaction. Visual data contains rich information about device use condition and tissue outcome, which may be used to evaluate the applicability as well as effectiveness of a procedure in minimally invasive surgeries (MIS). The images and instrument data (which may be captured by an electrosurgical unit (ESU)) may be recorded simultaneously. Depending on the instrument activation state (LOW for off and HIGH for on), the system may automatically record video streams and/or capture pre/intra/post energy application images. After the energy is delivered (e.g., where the instrument activation channel has returned to LOW), the system may query ESU to retrieve the latest electrical data file and save it together with video/image files to a specific location on a storage device. The video/image data may be used later to annotate instrument electrical data—providing “ground truth” for supervised learning that enables determination of relationships between a variety of sensory inputs and energy delivery outcomes. The term “annotate” refers to use conditions such as tissue type and size for example. The term “tag” refers to outcomes such as, for example, seal strength.

The terms “artificial intelligence,” “data models,” or “system learning” may include, but are not limited to, neural networks, recurrent neural networks (RNN), generative adversarial networks (GAN), Bayesian Regression and Inference, Naive Bayes, nearest neighbors, least squares, means, and support vector regression, among other data science and artificial science techniques.

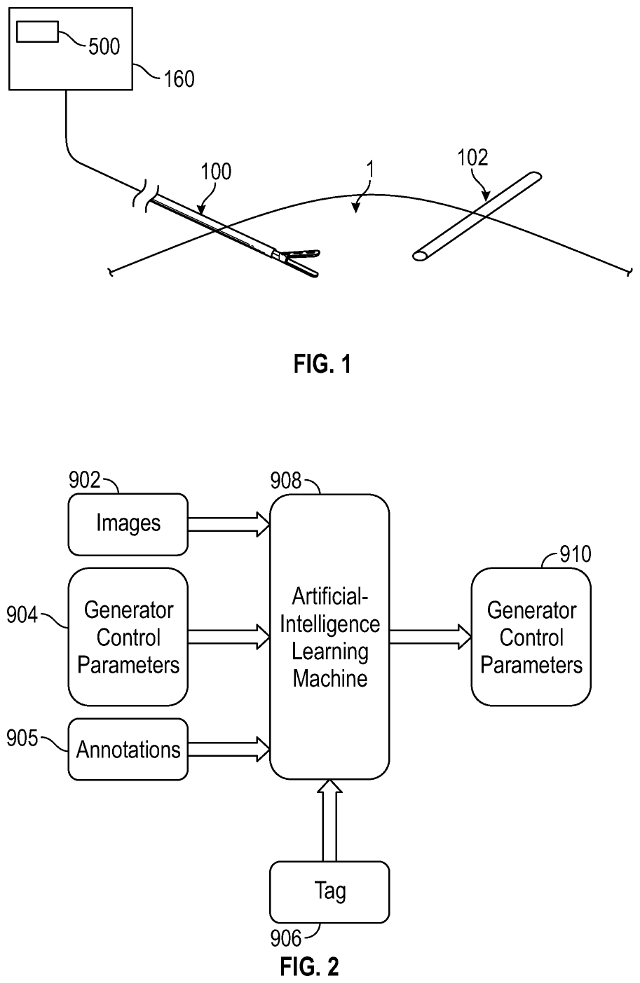

The term “application” may include a computer program designed to perform particular functions, tasks, or activities for the benefit of a user. Application may refer to, for example, software running locally or remotely, as a standalone program or in a web browser, or other software that would be understood by one skilled in the art to be an application. An application may run on a controller, e.g., controller 500 (FIG. 1) or on a user device, including for example, on a mobile device, an IoT device, or a server system.

Referring now to FIG. 1, an energy-based surgical system provided in accordance with the disclosure includes a generator 160, an energy-based surgical instrument, e.g., an endoscopic surgical forceps 100 for use in connection with endoscopic surgical procedures, and an endoscope 102. The endoscope 102 is used to capture images of a surgical site 1. Although endoscope 102 is illustrated, it is understood that other image capturing devices may be used, e.g., located on the energy-based surgical instrument. Aspects of the generator 160 will be described in more detail later herein.

With reference to FIG. 2, a block diagram of an artificial intelligence learning system 908 is shown in accordance with aspects of the disclosure. As explained in more detail below, the artificial intelligence learning system may leverage clinician input, e.g., annotations 905, as well as other tagged data, to facilitate determining whether or not a current generator configuration is suitable for achieving a successful outcome for a particular situation. Training of the artificial intelligence learning system 908 may use training data that includes images 902 and current generator control parameter values 904 as inputs to the artificial intelligence learning system 908. In various embodiments, the training data may be tagged with a tag 906, which persons skilled in the art will understand as a label that reflects some ground truth about the training data, thus enabling the artificial intelligence learning system 908 to determine whether the parameters applied in a particular situation resulted in a success (and, in embodiments, to what level of success) and to optimize the generator parameters for a given condition or set of conditions based thereon. In embodiments, the artificial intelligence learning system 908 may be implemented for use during a procedure to output generator control parameters 910 based on the input data and/or to output an indication regarding whether or not to maintain the current generator control parameters 910 based on the input data.

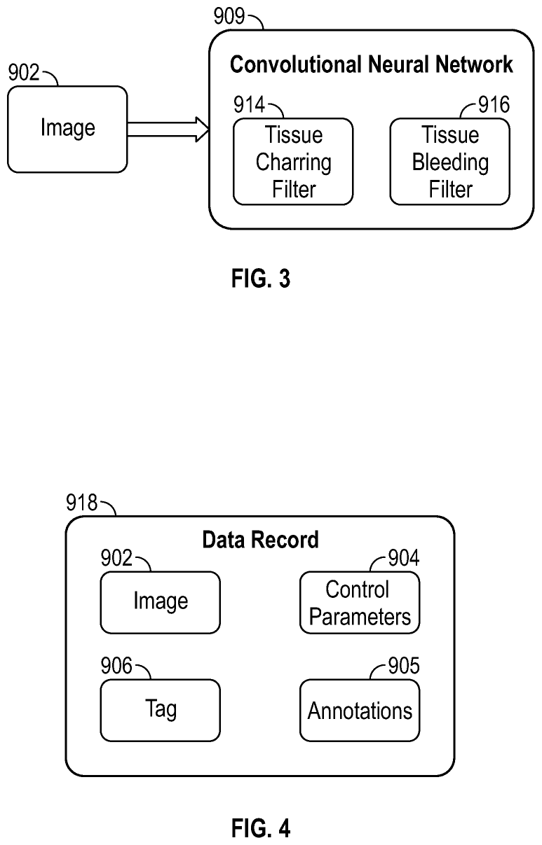

The images 902 that are input to the artificial intelligence learning system 908 may show tissue characteristics such as, for example, tissue bleeding and/or tissue charring, tissue color, tissue opacity, etc. The artificial intelligence learning system 908 may be configured to identify such characteristics in the image 902. With reference to FIG. 3, the artificial intelligence learning system may include a convolutional neural network 909 that has a filter configured to identify, for example, tissue charring 914 and/or a filter configured to identify tissue bleeding 916. Persons skilled in the art will understand how to implement and operate such convolutional neural networks 909 and filters. These and/or other tissue characteristics can be identified in the image 902 by the convolutional neural network 909 and can augment/supplement the input data to the artificial intelligence learning system 908 (FIG. 2). Other suitable traditional or artificial intelligence learning systems may be utilized

Referring to FIG. 4, a training data record 918 may include an image 902, associated stored generator control parameters 904, and an associated tag 906, which are used to train the artificial intelligence learning system 908. In various embodiments, the generator control parameters 904 may include, but are not limited to, for example, duration, power, ramp rate, frequency, or other generator parameters. In various embodiments, the training data record 918 may include data relating to annotations 905, such as tissue type, tissue size, steam/smoke/vapor, jaw closure, tissue sticking, bleeding, lateral thermal spread, tissue moisture, hydration, and/or tissue location within the patient's body, among other tissue characteristics. A person skilled in the art would understand how to determine tissue moisture, such as, for example, as described in U.S. Pat. No. 8,961,504. In various embodiments, the data relating to annotations 905 may be entered into the system manually, electronically from the patient's medical records, or automatically imported from other systems and/or instruments.

In various embodiments, as mentioned above, the image 902, the stored generator control parameter values 904, and the annotations 905, form training data. In various embodiments, the tag 906 relates to the training data 902-905 in a specific way. In particular, with respect to a particular application of energy to tissue based on generator control parameter values, the training data 902-905 is captured before such energy is applied to the tissue, and the tag 906 is applied after such energy is applied to the tissue. Thus, the training data 902-905 reflects a scenario that a clinician may face before energy is applied to a tissue, and the tag 906 reflects the outcome of such energy application to the tissue, e.g., whether it was a successful outcome or an unsuccessful outcome, a rating of the outcome, a burst pressure probability, etc. Training the artificial-intelligence learning system in the manner described above results in identifying correlations between the generator parameter inputs and energy delivery outcomes, and thereby allows the artificial intelligence learning system to learn from clinician experience.

It is contemplated that the training may be performed on a separate system, for example, GPU servers, simulation, etc., and the trained network would then be deployed to the energy-based surgical system.

Referring generally to FIGS. 1-4, as mentioned above, the artificial-intelligence learning machine 908 determines correlations between the input data 902-905 and the outcomes of energy-based surgical procedures, and thereby learns from clinician experience through the training process. In applying a trained artificial intelligence learning system 908 during a surgical procedure, the controller 500 outputs, from the artificial-intelligence learning system, an indication of whether to maintain a generator's current control parameter values based on tissue image 902, the current generator control values 904, and/or annotations 905. In various embodiments, the indicator may be implemented as a visual indication, such as an LED light or a display screen. Alternatively or additionally, the generator's current control parameter values may be automatically updated, energy delivery may be stopped, and/or other actions and/or indicators may be provided. In various embodiments, the generator 160 provides the visual indication. In various embodiments, the energy-based surgical instrument, e.g., forceps 100, provides the visual indication. In various embodiments, the image 902 may be captured before, during, and/or after energy delivery.

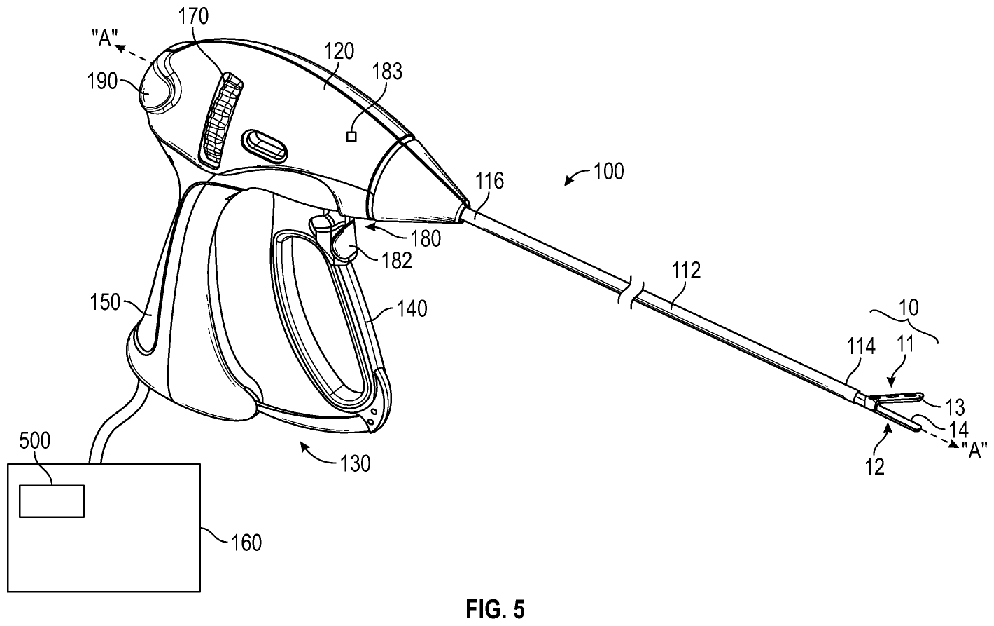

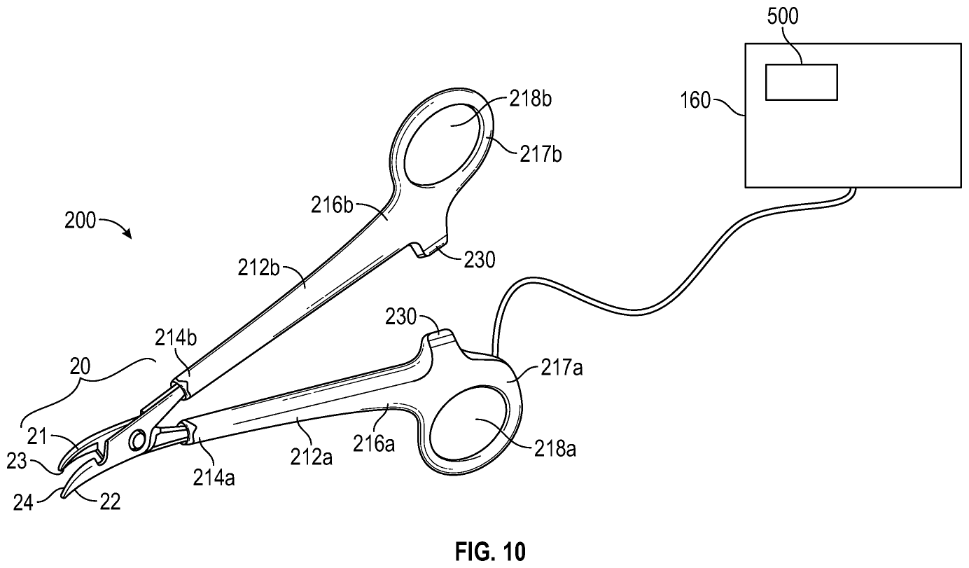

Referring now to FIG. 5, there is shown endoscopic surgical forceps 100 in more detail and generator 160 for use therewith. For the purposes herein, either endoscopic forceps 100, open forceps 200 (see FIG. 10), or any other suitable surgical instrument and/or system may be utilized in accordance with the disclosure. Obviously, different electrical and mechanical connections and considerations apply to each particular type of instrument and system; however, the aspects and features of the disclosure remain generally consistent regardless of the configuration of the instrument(s) or system(s) used therewith.

Endoscopic forceps 100 defines a longitudinal axis “A-A” and includes a housing 120, a handle assembly 130, a rotating assembly 170, a trigger assembly 180, and an end effector assembly 10. Forceps 100 further includes a shaft 112 having a distal end 114 configured to mechanically engage end effector assembly 10 and a proximal end 116 that mechanically engages housing 120. Forceps 100 may further include a surgical cable extending therefrom and configured to connect forceps 100 to generator 160 such that at least one of the electrically-conductive tissue treating surfaces 13, 14 of jaw members 11, 12 of end effector assembly 10 may be energized to treat tissue grasped therebetween, e.g., upon activation of activation switch 190.

With continued reference to FIG. 5, handle assembly 130 includes fixed handle 150 and a movable handle 140. Fixed handle 150 is integrally associated with housing 120 and handle 140 is movable relative to fixed handle 150. Rotating assembly 170 is rotatable in either direction about a longitudinal axis “A-A” to rotate end effector assembly 10 about longitudinal axis “A-A.” Housing 120 houses the internal working components of forceps 100.

End effector assembly 10 is shown attached at distal end 114 of shaft 112 and includes a pair of opposing jaw members 11 and 12. Each of jaw members 11 and 12 includes an electrically-conductive tissue treating surface 13, 14, respectively, configured to grasp tissue therebetween and conduct energy therethrough to treat, e.g., seal, tissue in a bipolar electrosurgical manner. End effector assembly 10 is designed as a unilateral assembly, i.e., where jaw member 12 is fixed relative to shaft 112 and jaw member 11 is movable relative to shaft 112 and fixed jaw member 12. However, end effector assembly 10 may alternatively be configured as a bilateral assembly, e.g., where both jaw member 11 and jaw member 12 are movable relative to one another and to shaft 112. In some embodiments, a knife assembly (not shown) is disposed within shaft 112, and a knife channel (not shown) is defined within one or both jaw members 11, 12 to permit reciprocation of a knife blade (not shown) therethrough, e.g., upon activation of trigger 182 of trigger assembly 180.

Continuing with reference to FIG. 5, movable handle 140 of handle assembly 130 is ultimately connected to a drive assembly (not shown) that, together, mechanically cooperate to impart movement of jaw members 11 and 12 between a spaced-apart position and an approximated position to grasp tissue between tissue treating surfaces 13 and 14 of jaw members 11, 12, respectively. As shown in FIG. 5, movable handle 140 is initially spaced-apart from fixed handle 150 and, correspondingly, jaw members 11, 12 are in the spaced-apart position. Movable handle 140 is depressible from this initial position to a depressed position corresponding to the approximated position of jaw members 11, 12.

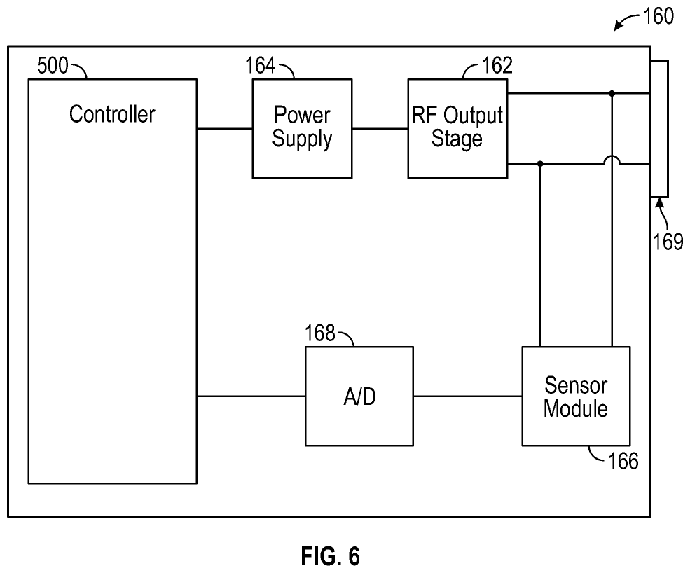

Referring also to FIG. 6, there is shown a block diagram of generator 160 in accordance with aspects of the disclosure. In the illustrated embodiment, the generator 160 includes a controller 500, a power supply 164, a radio-frequency (RF) energy output stage 162, a sensor module 166, and one or more connector ports 169 that accommodate various types of energy-based surgical instruments, e.g., endoscopic forceps 100 (FIG. 5) and open forceps 200 (see FIG. 10). The generator 160 can include a user interface (not shown), which permits a user to select and/or view various parameters for the generator 160, such as mode of operation and power setting. In various embodiments, the power setting can be specified by a user to be between zero and a power limit, such as, for example, five watts, thirty watts, seventy watts, or ninety-five watts.

The generator 160 may be any suitable type of generator to accommodate various types of energy-based surgical instruments (e.g., monopolar energy-based surgical instrument and bipolar electrosurgical instruments such as forceps 100, 200 (FIGS. 5 and 10, respectively)). The generator 160 may also be configured to operate in a variety of modes, such as ablation, cutting, coagulation, and sealing. The generator 160 may include a switching mechanism (e.g., relays) to switch the supply of RF energy among the connector ports 169 to which various energy-based surgical instruments may be connected. For example, when an energy-based surgical instrument, e.g., forceps 100 (FIG. 5) or forceps 200 (FIG. 10), is connected to the generator 160, the switching mechanism switches the supply of RF energy to the connector port 169. In embodiments, the generator 160 may be configured to provide RF energy to a plurality of instruments simultaneously.

In various embodiments, the sensor module 166 includes a plurality of sensors, e.g., an RF current sensor and an RF voltage sensor. Various components of the generator 160, namely, the RF output stage 162 and the RF current and voltage sensors of sensor module 166 may be disposed on a printed circuit board (PCB). The RF current sensor of sensor module 166 may be coupled to the active terminal and provides measurements of the RF current supplied by the RF output stage 162. In embodiments, the RF current sensor of sensor module 166 may be coupled to the return terminal. The RF voltage sensor of sensor module 166 is coupled to the active and return terminals and provides measurements of the RF voltage supplied by the RF output stage 162. In embodiments, the RF current and voltage sensors of sensor module 166 may be coupled to active and return leads which interconnect the active and return terminals, and the RF output stage 162.

The sensed voltage and current from sensor module 166 are fed to analog-to-digital converters (ADCs) 168. The ADCs 168 sample the sensed voltage and current to obtain digital samples of the voltage and current of the RF output stage 162. The digital samples are processed by the controller 500 and used to generate a control signal to control the DC/AC inverter of the RF output stage 162 and the preamplifier. The ADCs 168 communicate the digital samples to the controller 500 for further processing.

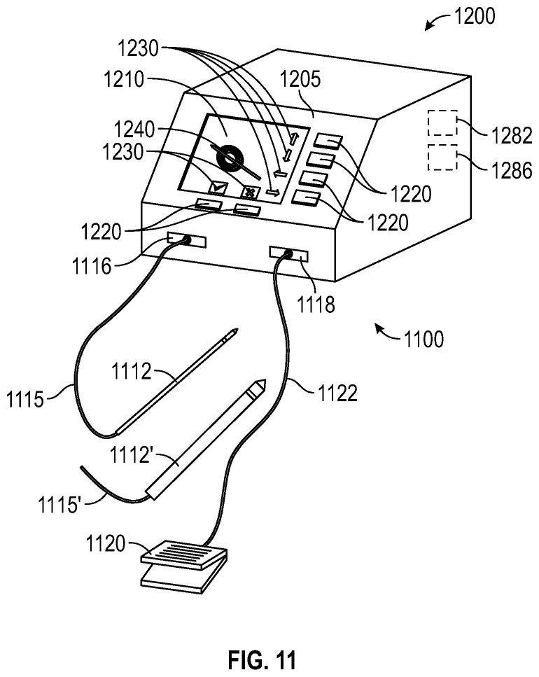

Continuing with reference to FIGS. 5 and 6, in various embodiments, the controller 500 may collect data relating to the generator 160, including time, power, and/or impedance. For example, in use, tissue is grasped between electrically conductive tissue treating surfaces 13, 14 of jaw members 11, 12 (or jaw members 21, 22 of forceps 200 (FIG. 11)) and electrosurgical (RF) energy is conducted between tissue treating surfaces 13, 14 and through tissue to heat and thereby treat tissue. During such tissue treatment, the sensor circuitry, e.g., sensor module 166, of the generator 160 may sense parameters of the tissue and/or energy such as, for example, impedance and power, and/or may supply data from which impedance and/or power can be derived such as for example, time, voltage, and/or current data. It is contemplated that jaw force or pressure may also be sensed or determined. This may occur as a snapshot or over a time interval and may be determined at the beginning of tissue treatment, e.g., at or within 250 ms of initiation of tissue treatment. The sensed data may include, for example, time that the power is applied for, power applied to the tissue, and/or impedance of the tissue. This sensed data obtained by the sensor circuitry is relayed to the controller 500 (via the ADC's 168, in embodiments) for further processing, as detailed below.

In various embodiments, the controller 500 uses the images, the control parameter values, and the annotations as training data for an artificial-intelligence learning system. In various embodiments, training and machine learning may be performed by a computing device outside of the generator 160, and the result of the machine learning may be communicated to the controller 500 of generator 160. In various embodiments, the controller 500 communicates the determined generator control parameter configuration that was output from the machine learning algorithm to a computing device, e.g., of controller 500. In various embodiments, the artificial intelligence learning system may use supervised learning, unsupervised learning, and/or reinforcement learning. In various embodiments, the neural network may include a temporal convolutional network, a fully connected network, or a feed-forward network.

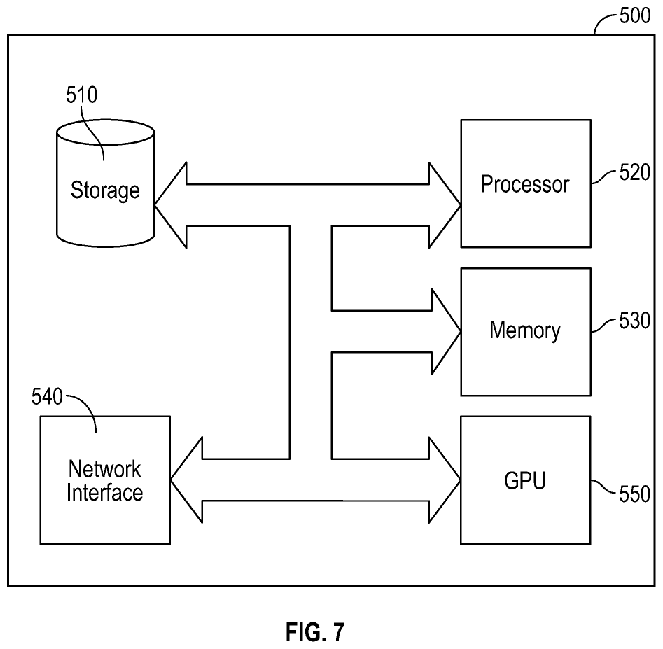

Referring to FIG. 7, a block diagram of controller 500 is shown. The controller 500 includes a processor 520, a computer-readable storage medium (e.g., a storage device 510), and/or a memory 530. In various embodiments, the processor 520 may be another type of processor such as, without limitation, a digital signal processor, a microprocessor, an ASIC, a field-programmable gate array (FPGA), or a central processing unit (CPU). In various embodiments, the controller 500 may include one or more graphics processing units (GPU) 550. The GPU 550 may be used for executing various machine learning algorithms such as those provided in accordance with the disclosure, as detailed below.

The memory 530, may be a volatile type memory, e.g., RAM, or a non-volatile type memory, e.g., flash media, disk media, etc. The memory 530 stores suitable instructions, to be executed by the processor 520, for receiving the sensed data, e.g., sensed data from sensor module 166 via ADCs 168 (see FIG. 6), accessing storage device 510 of the controller 500, determining one or more tissue parameters, e.g., tissue temperature, based upon the sensed data and information stored in storage device 510, and providing feedback based upon the determined tissue parameters. Storage device 510 of controller 500 stores one or more algorithms and/or models configured to estimate one or more tissue parameters, e.g., tissue temperature, based upon the sensed data received from sensory circuitry, e.g., from sensor module 166 via ADCs 168 (see FIG. 6). Although illustrated as part of generator 160, it is also contemplated that controller 500 be remote from generator 160, e.g., on a remote server, and accessible by generator 160 via a wired or wireless connection. In embodiments where controller 500 is remote, it is contemplated that controller 500 may be accessible by and connected to multiple generators 160.

In various embodiments, the memory 530 can be separate from the controller 500 and can communicate with the processor 520 through communication buses of a circuit board and/or through communication cables such as serial ATA cables or other types of cables. In various embodiments, the controller 500 may include a network interface 540 to communicate with other computers or a server.

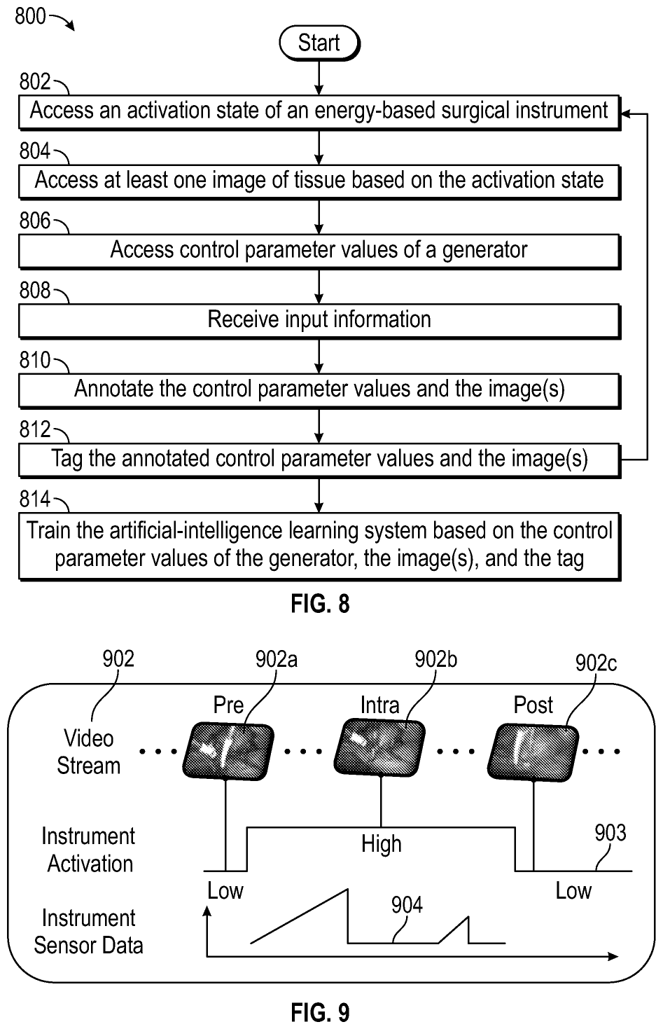

Referring now to FIG. 8, there is shown a flow diagram of a computer-implemented method 800 for method for controlling an energy-based surgical system. Persons skilled in the art will appreciate that one or more operations of the method 800 may be performed in a different order, repeated, and/or omitted without departing from the scope of the disclosure. In various embodiments, some or all of the operations in the illustrated method 800 can operate using an energy-based surgical system, e.g., instrument 100 or 200 (see FIGS. 5 and 10) and the generator 160 (see FIGS. 2 and 3). Other variations are contemplated to be within the scope of the disclosure. The operations of FIG. 8 will be described with respect to a controller, e.g., controller 500 of generator 160 (FIGS. 6 and 7), but it will be understood that the illustrated operations are applicable to other systems and components thereof as well.

Initially, at step 802, the controller 500 may access an activation state of an energy-based instrument captured before, during, and/or after an energy-based surgical procedure (FIG. 9). In various embodiments, the value of the activation channel may be, for example, low or high indicating that the electrosurgical energy is being applied or not. For example, prior to energy application, the activation state of the energy-based instrument may be low (FIG. 9).

At step 804, the controller 500 may access an image of the instrument and/or tissue captured before, during, and/or after an energy-based surgical procedure. In various embodiments, the image may be a video or a still image. For example, the image data may contain rich information about tissue, energy-based surgical instrument use condition, and/or outcome of the energy-based tissue treatment. In various embodiments, a data logger, consisting of a video processor and a storage unit may be used to capture and process the image.

At step 806, the controller 500 accesses control parameter values of a generator, e.g., generator 160, which provides energy to an energy-based surgical instrument. In various embodiments, the parameters may include time, slope, power, and/or impedance. In various embodiments, the controller 500 stores the control parameter values.

At step 808, the controller 500 receives input information (e.g., from a clinician and/or from another source). In various embodiments, the information may include tissue type, tissue size, field condition, presence of steam, a presence of smoke, presence of vapor, jaw closure, tissue sticking, bleeding, and/or lateral thermal spread. For example, the capture and annotation of video/image data along with control parameters of the generator 160 provides “context” for the energy-tissue interaction.

At step 810, the controller 500 annotates the stored control parameter values and the image(s) based on the received information. For example, a pre-energy application image may be captured based on the value of the instrument activation channel being low, e.g., where energy is not being applied. The pre-energy application image may be annotated, for example, as follows: tissue type: uterine pedicle; tissue size: large; and field condition: dry.

At step 812, controller 500 tags the annotated control parameter values and the image(s) for training an artificial intelligence learning system.

At step 814, the controller 500 may train the artificial-intelligence learning system based on the stored control parameter values of the generator 160, the image(s), and the tag(s). In various embodiments, the artificial intelligence learning system may be trained by the controller 500 or remotely. For example, the training may include unsupervised learning, supervised learning, and/or reinforcement learning.

In various embodiments, the artificial intelligence learning system processes the image and the stored control parameters, and/or the annotations, to provide an output relating to whether or not to maintain the current generator control parameters, and/or to automatically update generator control parameters. In various embodiments, the control parameters may include time, slope, power, and/or impedance. In various embodiments, the image, the control parameters, and/or the annotations may provide “ground truth” for supervised learning. In various embodiments, the artificial intelligence learning system, can predict outcomes such as seal strength, using supervised learning. In various embodiments, the artificial intelligence learning system, can be used to understand use conditions such as the occurrence of extra-large vessels in a given patient cohort using unsupervised learning.

In various embodiments, the controller 500 adjusts, automatically by the generator 160, the control parameters to provide adjusted energy based on the output of the artificial-intelligence learning system. In various embodiments, the controller 500 may deliver energy, by an energy-based surgical instrument configured to deliver energy to tissue, using the adjusted control parameters. In various embodiments, the controller 500 may store settings of the energy-based surgical instrument based on the adjusted control parameters, in a memory of the generator 160. Thus, with the artificial intelligence learning system having been trained as detailed above, the system can determine whether to maintain a generator control parameter configuration for generating energy to treat tissue.

Referring now to FIGS. 7 and 9, an illustration showing a relationship between a video stream, instrument activation and instrument sensor data is shown. For example, controller 500 accesses the value of the instrument activation 903, which may be initially low (grasping tissue in an un-energized state, for example). The controller 500 then captures or receives images via the video stream 902 and determines, based on the instrument activation 903 state, that the image is a pre-energy application image 902a. Accordingly, the controller 500 accesses the control parameters of the generator 160 and associates them with the image(s) 902a. In another example, the controller 500 may access the instrument activation 903 state, which may be high, (indicating that energy is being applied). The controller 500 then captures images via the video stream 902 and determines that based on that instrument activation 903 state that the image is captured during an energy-based tissue treatment 902b, e.g., during application of energy to grasped tissue. Accordingly the controller 500 accesses the control parameters of the generator 160 and associates them with the image(s). In yet another example, the controller 500 may subsequently access the instrument activation 903 state, which may have returned to low. However, the controller 500 has previously recorded a high state for the instrument activation 903 state. The controller 500 then captures images via the video stream 902 and determines that, based on that current low state and the previous high state, the image is captured after energy application 902c. Accordingly the controller 500 accesses the control parameters of the generator 160 and associates them with the image(s).

Referring now to FIG. 10, open forceps 200 is shown, including two elongated shafts 212a and 212b, each having a proximal end 216a and 216b, and a distal end 214a and 214b, respectively. Aspects of the disclosure with reference to FIG. 1-8, apply to the forceps 200 of FIG. 10 as well as any other suitable energy-based surgical instrument. In particular, capturing an image of the surgical site may be accomplished using the image capturing device located on a portion of the forceps 200. In various embodiments, the camera may be separate from the forceps 200, such as incorporated into an endoscope.

Forceps 200 is configured for use with an end effector assembly 20 that is similar to end effector assembly 10 of forceps 100 (see FIG. 5). More specifically, end effector assembly 20 is attached to distal ends 214a and 214b of shafts 212a and 212b, respectively, and includes a pair of opposing jaw members 21 and 22 that are movable relative to one another. Each shaft 212a and 212b includes a handle 217a and 217b disposed at the proximal end 216a and 216b thereof. Each handle 217a and 217b defines a finger hole 218a and 218b therethrough for receiving a finger of the user. As can be appreciated, finger holes 218a and 218b facilitate movement of shafts 212a and 212b relative to one another from an open position, wherein jaw members 21 and 22 are disposed in spaced-apart relation relative to one another, to a closed position, wherein jaw members 21 and 22 cooperate to grasp tissue therebetween.

A ratchet 230 may be included for selectively locking jaw members 21 and 22 of forceps 200 relative to one another at various different positions. It is envisioned that ratchet 230 may include graduations or other visual markings that enable the user to easily and quickly ascertain and control the amount of closure force desired between the jaw members 21 and 22.

With continued reference to FIG. 10, one of the shafts may be adapted to receive a surgical cable configured to connect forceps 200 to generator 160 such that at least one of the electrically-conductive tissue treating surfaces 23, 24 of jaw members 21, 22, respectively, of end effector assembly 20 may be energized to treat tissue grasped therebetween.

Similar to forceps 100 (FIG. 5), forceps 200 may further include a knife assembly (not shown) disposed within either of shafts 212a, 212b and a knife channel (not shown) defined within one or both jaw members 21, 22 to permit reciprocation of a knife blade (not shown) therethrough.

Referring now to FIG. 11, illustrated is a microwave ablation system 1100 in accordance with the disclosure. The system 1100 includes a generator 1200, microwave antenna probe 1112 operably coupled by a cable 1115 via connector 1116 to the generator 1200, and an actuator 1120 (which may be a footswitch, a hand-switch, or any other suitable actuator). Actuator 1120 is operably coupled by a cable 1122 via connector 1118 to generator 1200. Cable 1122 may include one or more electrical conductors for conveying an actuation signal from actuator 1120 to generator 1200.

At least one additional or alternative microwave antenna probe 1112′ may be included with microwave ablation system 1100 that may have characteristics distinct from that of microwave antenna probe 1112. For example, without limitation, microwave antenna probe 1112 may be a 12-gauge probe suitable for use with energy of about 915 MHz, while microwave antenna probe 1112′ may be a 14-gauge probe suitable for use with energy of about 915 MHz. Other probe variations are contemplated within the scope of the disclosure, for example, without limitation, a 12-gauge operable at 2450 MHz, and a 14 gauge operable at 2450 MHz. In use, the surgeon may interact with user interface 1205 of generator 1200 to preview operational characteristics of available probes 1112, 1112′, and to choose a probe for use.

Generator 1200 includes a generator module 1286 that is configured as a source of microwave energy and is disposed in operable communication with processor 1282. In embodiments, generator module 1286 is configured to provide energy of about 915 MHz. Generator module 1286 may also be configured to provide energy of about 2450 MHz (2.45 GHz.). The disclosure contemplates embodiments wherein generator module 1286 is configured to generate a frequency other than about 915 MHz or about 2450 MHz, and embodiments wherein generator module 1286 is configured to generate variable frequency energy. Probe 1112 is operably coupled to an energy output of generator module 1286.

Generator assembly 1200 also includes user interface 1205, which may include a display 1210 such as, without limitation, a flat panel graphic LCD display, adapted to visually display at least one user interface element 1230, 1240. In embodiments, display 1210 includes touchscreen capability, e.g., the ability to receive input from an object in physical contact with the display, such as without limitation a stylus or a user's fingertip, as will be familiar to the skilled practitioner. User interface elements 1230, 1240 may have a corresponding active region, such that, by touching the screen within the active region associated with a user interface element 1230, 1240, an input associated with the user interface element is received by the user interface 1205.

User interface 1205 may additionally or alternatively include one or more controls 1220, which may include without limitation a switch (e.g., pushbutton switch, toggle switch, slide switch) and/or a continuous actuator (e.g., rotary or linear potentiometer, rotary or linear encoder.) In embodiments, a control 1220 has a dedicated function, e.g., display contrast, power on/off, and the like. Control 1220 may also have a function that may vary in accordance with an operational mode of the ablation system 1100. A user interface element 1230 may be positioned substantially adjacently to control 1220 to indicate the function thereof. Control 1220 may also include an indicator, such as an illuminated indicator (e.g., a single-colored or variably-colored LED indicator).

For example, the processor 1282 may access an activation state of a generator 1200 captured before, during, and/or after an energy-based surgical procedure (see FIG. 9). For example, during energy application, the activation state of the energy-based instrument may be high (FIG. 9) indicating that the electrosurgical energy is being applied. Next, the processor 1282 may access an image of a tissue captured before, during, and/or after an energy-based surgical procedure. For example, the image of the tissue may be captured during energy application. Next, the processor 1282 accesses control parameter values of the generator 1200, such as time, slope, power, and/or impedance. Next, the processor 1282 receives input information (e.g., from a clinician and/or from another source). For example, the input information may indicate that the tissue size is large, and that there is bleeding, and lateral thermal spread. Next, in various embodiments, the artificial intelligence learning system may process the image and the control parameters, and/or the annotations, to provide an output relating to whether or not to maintain the current generator control parameters, and/or to automatically update generator control parameters. For example, reducing the output power of the generator 1200.

It is contemplated that the instruments and systems detailed herein may be part of a robotic surgical system as opposed to a handheld instrument. Thus, aspects, as described herein, apply to such a robotic surgery system as well.

From the foregoing and with reference to the various figure drawings, those skilled in the art will appreciate that certain modifications can also be made to the disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A computer-implemented method for use with an energy-based surgical system, comprising:
accessing an activation state of an energy-based surgical instrument;
accessing at least one image of tissue based on the activation state;
accessing control parameter values of a generator configured to provide energy to the energy-based surgical instrument;
storing the control parameter values;
receiving input information;
annotating the stored control parameter values and the at least one image based on the received information; and
tagging the annotated control parameter values and the annotated at least one image.

2. The computer-implemented method of claim 1, wherein the instrument activation state is at least one of prior to energy delivery, during energy delivery, or after energy delivery.

3. The computer-implemented method of claim 2, wherein the at least one image is captured at least one of prior to energy delivery, during energy delivery, or after energy delivery.

4. The computer-implemented method of claim 2, wherein in a case where the at least one image is captured prior to energy delivery, the annotation includes at least one of tissue type, tissue size, or field condition.

5. The computer-implemented method of claim 2, wherein in a case where the at least one image is captured during energy delivery, the annotation includes at least one of a presence of steam, a presence of smoke, a presence of vapor, or jaw closure.

6. The computer-implemented method of claim 2, wherein in a case where the at least one image is captured after energy delivery, the annotation includes at least one of tissue sticking, bleeding, or lateral thermal spread.

7. The computer-implemented method of claim 1, wherein the tagging includes providing a tag for training an artificial intelligence learning system, and further comprising:
training the artificial intelligence learning system based on the stored control parameter values of the generator, the at least one image, and the tag.

8. The computer-implemented method of claim 6, wherein the output of the artificial-intelligence learning system relates to a predicted outcome of applying the energy to tissue based on the stored control parameter values.

9. The computer-implemented method of claim 6, wherein the artificial intelligence learning system includes a convolutional neural network that processes the at least one image.

10. The computer-implemented method of claim 1, further comprising:
processing the at least one image, the stored control parameter values, and the annotations by an artificial intelligence learning system to provide an output relating to a configuration of the control parameter values;
providing an indication to a clinician based on the output, the indication indicating whether to maintain the control parameter values; and
in a case where the indication indicates not to maintain the control parameter values, providing adjusted control parameter values for the generator based on the output of the artificial-intelligence learning system.

11. The computer-implemented method of claim 10, further comprising:
automatically adjusting the control parameters based on the output of the artificial-intelligence learning system; and
providing an indication to a clinician that the control parameters have been automatically adjusted.

12. The computer-implemented method of claim 1, further comprising:
outputting the energy to an energy-based surgical instrument;
applying the energy to tissue using the energy-based surgical instrument; and
receiving information from the clinician regarding an outcome of applying the energy to tissue.

13. The computer-implemented method of claim 1, wherein the at least one image includes at least one of a video image or a still image.

14. An energy-based surgical system comprising:
an image capturing device configured to capture at least one image of tissue; and
a generator configured to provide energy to an energy-based surgical instrument, the generator configured to execute instructions to perform a method including:
accessing an activation state of an energy-based surgical instrument;
accessing the at least one image of tissue;
accessing control parameter values of the control parameters;
storing the control parameter values;
receiving input information;
annotating the stored control parameter values and the at least one image based on the received information; and
tagging the annotated control parameter values and the at least one image.

15. The energy-based surgical system of claim 12, wherein the instrument activation state is at least one of prior to energy delivery, during energy delivery or after energy delivery.

16. The energy-based surgical system of claim 13, wherein the at least one image is captured at least one of prior to energy delivery, during energy delivery, or after energy delivery.

17. The energy-based surgical system of claim 13, wherein in a case where the at least one image is captured prior to energy delivery, the annotation includes at least one of tissue type, tissue size, or field condition.

18. The energy-based surgical system of claim 13, wherein in a case where the at least one image is captured during energy delivery, the annotation includes at least one of a presence of steam, a presence of smoke, presence of vapor, or jaw closure.

19. The energy-based surgical system of claim 13, wherein in a case where the at least one image is captured after energy delivery, the annotation includes at least one of tissue sticking, bleeding, or lateral thermal spread.

20. A non-transitory storage medium that stores a program causing a computer to execute a method for an energy-based surgical system, the method comprising:
accessing an activation state of an energy-based surgical instrument;
accessing at least one image of tissue;
accessing control parameter values of a generator configured to provide energy to the energy-based surgical instrument;
storing the control parameter values;
receiving input information;
annotating the stored control parameter values and the at least one image based on the received information; and
tagging the annotated control parameter values and the annotated at least one image.
